(19) United States
(12) Patent Application Publication
(10) Pub. No.: US 2026/0151220 A1
(43) Pub. Date: Jun. 4, 2026

(54) SYSTEM FOR PROMOTING BIOLOGICAL ATTACHMENT OF CONNECTIVE TISSUE TO BONE IMPLANTS

(71) Applicant: SIGNATURE ORTHOPAEDICS EUROPE LTD, Dublin (IE)

(72) Inventors: Joshua PORTER, Dublin (IE); Declan BRAZIL, Lane Cove West (AU)

(21) Appl. No.: 18/865,236

(22) PCT Filed: May 29, 2024
(86) PCT No.: PCT/AU2024/050556

§ 371 Date: Nov. 12, 2024

(30) Foreign Application Priority Data
Jun. 1, 2023 (AU) 2023901725

(51) Int. Cl. A61F 2/08; A61L 27/56

(52) U.S. Cl. CPC A61F 2/0811; A61L 27/56

(57) ABSTRACT

The described system designed to promote the biological attachment of fibrous connective tissue to a bone implant features a bone implant with a scaffold defining a porous attachment surface, a suction port, and a manifold that distributes a negative pressure gradient across the attachment surface. The system also includes an extracorporeal vacuum pump and a percutaneous suction hose connecting the suction port of the implant to the vacuum pump. The implant is placed in the patient's body with connective tissue applied over the attachment surface, and the suction hose is attached and connected to the vacuum pump. The vacuum pump, operating outside the body, creates a negative pressure gradient over an extended period, promoting the growth of slow growing Sharpey's fibres for superior biological integration. A highly vascularised tissue graft, such as a pedicled muscle flap, may be applied adjacent to the avascular tendon to enhance endogenous growth factor migration.

### FIELD OF THE INVENTION

The present invention relates to the field of orthopaedic and biomedical engineering, specifically to systems and methods for promoting the biological attachment of fibrous connective tissue to bone implants. This invention addresses the enhancement of connective tissue integration with implants using negative pressure gradients to facilitate the growth of Sharpey's fibres, for improved biomechanical stability and long-term success of orthopaedic reconstructions.

### BACKGROUND OF THE INVENTION

Tendon or ligament connective tissue often needs to be attached to bone implants during salvage reconstructions, which are necessitated by significant bone loss due to trauma, cancer, or similar conditions. This process requires securely affixing the connective tissue to the implant to ensure it can withstand the mechanical forces applied during movement and load-bearing activities. To achieve this secure attachment, various mechanical fasteners, including bone attachment screws, anchors, and other fixation devices, are employed.

However, mechanical affixation is often inferior to the natural fibrous enthesis. Fasteners may not replicate the complex biomechanical properties of the natural tendon-bone interface, which can sometimes result in the physical detachment of the connective tissue from the mechanical fasteners, leading to implant failure and the need for revision surgery. These complications highlight the ongoing need for advancements in biomaterials and techniques to enhance the integration and durability of connective tissue attachments in orthopaedic reconstructions.

### SUMMARY OF THE DISCLOSURE

The system described is designed for promoting biological attachment of fibrous connective tissue to a bone implant.

The system comprises a bone implant featuring a scaffold that defines a porous attachment surface, a suction port, and a manifold that interfaces between the scaffold and the suction port to distribute a negative pressure gradient across the attachment surface. Additionally, the system includes an extracorporeal vacuum pump and a percutaneous suction hose that interfaces the suction port of the implant with the vacuum pump.

In use, the implant is implanted into a patient's body with the connective tissue applied over the attachment surface. The suction hose is then attached to the suction port, and connected to the vacuum pump, such as by passing through a small wound in the patient's epidermis.

The vacuum pump, operated outside the body, creates a negative pressure gradient across the attachment surface over a long period, thereby promoting growth of Sharpey's fibres for superior biological integration of the connective tissue with the scaffold. After the specified period, the percutaneous suction hose is detached from the suction port.

The current system is designed to provide a constant negative pressure gradient for an extended period after the implant is placed, using a vacuum pump operated outside the patient's body and connected via a suction hose. This negative pressure gradient encourages endogenous growth factors to migrate into the avascular connective tissue, promoting the growth of Sharpey's fibres, which are specialised collagen fibres that play a crucial role in anchoring tendons and ligaments to the bone, ensuring a strong and stable connection.

The present system uses the negative pressure gradient to encourage growth and embedding of embedding Sharpey's fibres deeply into the porous attachment surface to provide significant tensile strength which helps withstand the mechanical forces exerted during movement.

The negative pressure gradient is maintained across the attachment surface for an extended period (such as several weeks or more) to allow for the slow growing Sharpey's fibres to penetrate into the porous surface of the implant, providing superior biomechanical attachment compared to what can be achieved using mechanical fasteners.

Given the typically avascular nature of the connective tissue which slows or hinders ingrowth, the system preferably applies a highly vascularised tissue graft, such as a pedicled muscle flap, adjacent to the avascular tendon. Which, under negative pressure, serves as a vascular source for healing cells and regenerative growth factors and cytokines (such as Platelet-derived growth factor (PDGF), Fibroblast growth factor (FGF), vascular endothelial growth factor (VEGF) and others) that migrate into the avascular connective tissue, enhancing the growth of Sharpey's fibres and improving the overall integration and stability of the implant.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a system for promoting biological attachment of fibrous connective tissue to a bone implant;

FIG. 2 shows a bone implant configured as a knee implant;

FIG. 3 shows a suction port of the implant;

FIG. 4 shows a cross section of the suction port of FIG. 3;

FIG. 5 shows a cross-sectional view of an attachment surface of the implant comprising a series of fenestrated needles;

FIG. 6 shows a cross-sectional view of an attachment surface of the implant comprising a series of cannulated needles;

FIG. 7 shows an embodiment of a knee implant with a fixation clamp;

FIG. 8 shows a top view of the implant of FIG. 7;

FIG. 9 shows a cross-sectional view of the implant of FIG. 7;

FIG. 10 shows a cross-sectional view of a hip implant;

FIG. 11 shows a front view of the system in accordance with an embodiment;

FIG. 12 shows a cross-sectional view of the system of FIG. 11;

FIG. 13 shows a knee reconstruction using the present system;

FIG. 14 shows a hip reconstruction using the present system; and

FIG. 15 shows shoulder reconstruction using the present system.

### DESCRIPTION OF EMBODIMENTS

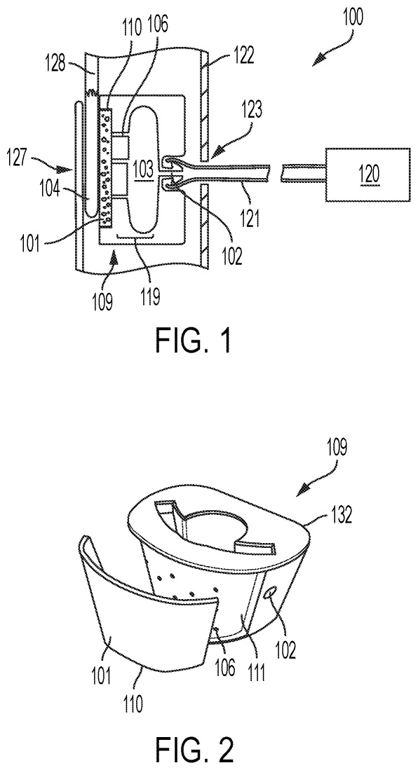

FIG. 1 shows a system 100 for promoting the biological attachment of fibrous connective tissue 104 to a bone implant 109. The connective tissue 104 may be a tendon attaching muscle 128. The implant 109 comprises a scaffold 110 defining a porous attachment surface 101. The implant 109 further includes a suction port 102 and a manifold 119 interfacing the scaffold 110 and the suction port 102. The manifold 119 may comprise an internal chamber 103 and a plurality of ducts 106 interfacing the chamber 103 and the scaffold 110.

The system 100 further comprises an extracorporeal vacuum pump 120, meaning that the vacuum pump 120 is located and operated outside the patient's body for some time after the implant 109 is installed. The system 100 also includes a percutaneous suction hose 121 interfacing the suction port 102 of the implant 109 with the vacuum pump 120.

The implant 109 is configured for implantation with the connective tissue 104 applied over the attachment surface 101. Mechanical fasteners may be used to hold the connective tissue 104 in place. Preferably, a vascular tissue graft 127 is applied over the connective tissue 104 to promote the migration of endogenous growth factors from the vascular tissue graft 127 into the avascular connective tissue 104. The vascular tissue graft 127 may be a pedicled muscle flap taken from an adjacent muscle, such as the calf muscle or gluteus maximus, depending on the type of reconstruction.

The suction hose 121 is attached to the suction port 102 during surgery, and the wound is closed with the suction hose 121 connected to the vacuum pump 120 located outside the patient's body. In the embodiment shown, the suction hose 121 may pass through a small wound 123 in the patient's epidermis 122, which may be sterilised and covered with dressing if necessary.

The pump 120 is then operated for a period to induce a negative pressure gradient across the attachment surface 101 to promote the biological integration of the connective tissue 104 to the scaffold 110. The negative pressure draws endogenous growth factors into the avascular connective tissue 104, thereby promoting the growth of Sharpey's fibres, which embed themselves into the porous attachment surface 101 to affix the connective tissue 104. The pump 120 may be operated for more than one week, preferably several weeks, to allow for adequate growth of these slow-growing collagenous fibres.

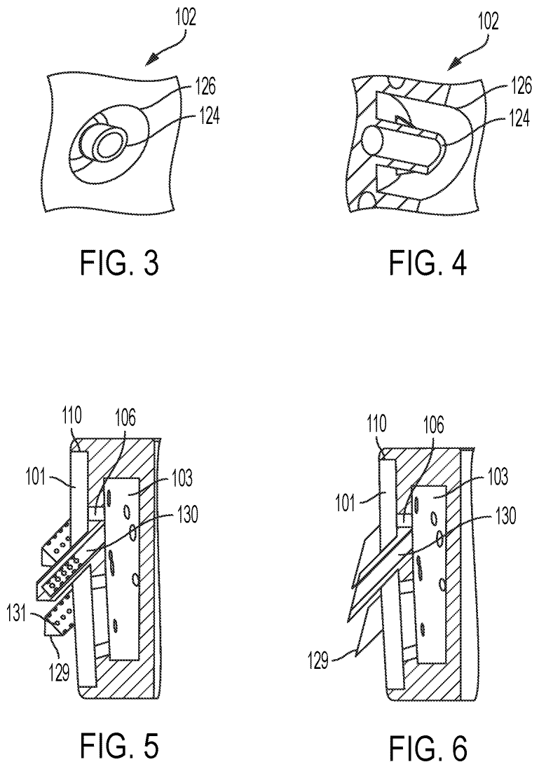

After the period, the suction hose 121 is disconnected from the implant 109, leaving the implant 109 in place. FIGS. 3 and 4 show an embodiment wherein the suction port 102 comprises a hose barb 124 which engages the inserted end of the suction hose 121. Preferably, the barb 124 is entirely concealed within a surrounding recess 126. The barb 124 is designed so that after the period, the delivery hose 121 can be pulled off the barb 124. The small wound 123 can then heal over.

The scaffold 110 is preferably made from ceramics, metals or alloys, polymers, or composites thereof.

FIGS. 5 and 6 show an embodiment wherein the fastener comprises a series of needles 129 which penetrate and mechanically engage the connective tissue 104. These needles 129 may be angled away with respect to the direction of force applied by the connective tissue 104. Preferably, the needles 129 define internal lumens 130 forming part of the manifold 119. According to the embodiment of FIG. 5, the needles 129 are fenestrated in that they comprise a series of side pores 131. According to the embodiment of FIG. 6, the needles 129 are cannulated.

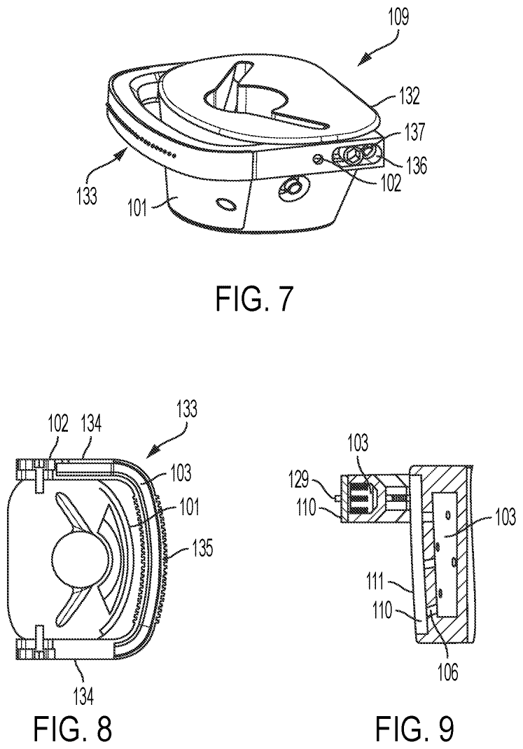

FIGS. 7-9 show an embodiment wherein the implant 109 comprises a main body 132 and a clamp 133 attachable thereto and configured to clamp across the connective tissue 104 against the attachment surface 101. In the embodiment shown, the clamp 133 is substantially U-shaped, comprising side arms 134 and a cross arm 135 which spans across the attachment surface 101. The side arms 134 may define slots 136 for adjustable engagement of a set screw 137 of the body 132 to account for different thicknesses of connective tissue 104.

The clamp 133 may itself define a manifold 119 configured to distribute a negative pressure gradient across the surface of the clamp 133. In this regard, the clamp 133 may comprise an independent suction port 102 to which a suction hose 121 is attached and an internal cavity 103 interfacing a scaffold 110. The scaffold 110 may similarly comprise a series of needles 129 for mechanical fixation and promoting endogenous growth factor migration. In the embodiment shown, the scaffold 110 is on an exterior surface of the clamp 133 but may also be on an internal surface of the clamp 133.

FIG. 2 shows an embodiment wherein the implant 109 is designed as a knee implant. The main body 132 may be metallic and may define a recess 111 into which a block of scaffold 110 is inserted and retained. The ducts 106 of the manifold 119 may be spread across an interior surface of the recess 111 to distribute the negative pressure gradient across the scaffold 110.

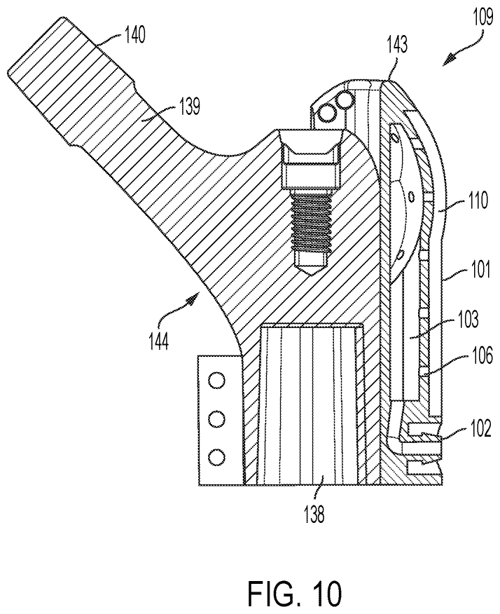

FIG. 10 shows an embodiment wherein the implant 109 is designed as a hip implant and may comprise a socket 138 for retention of an intramedullary stem and a neck 139 with a distal Morse taper 140 for engaging a spherical head 142. The implant 109 comprises the scaffold 110, attachment surface 101, manifold 119 (comprising the internal chamber 103 and ducts 106), and suction port 102 on a lateral side thereof. In the embodiment shown, the implant 109 comprises a main portion 144 and a separable augment 143 attachable to the main portion 144 and wherein the augment 143 comprises the manifold 119, suction port 102, and scaffold 110. In this regard, the augment 143 may comprise an internal channel slidably engaging the conventional implant 144 therein. The augment 143 may thus be attached to conventional implant components.

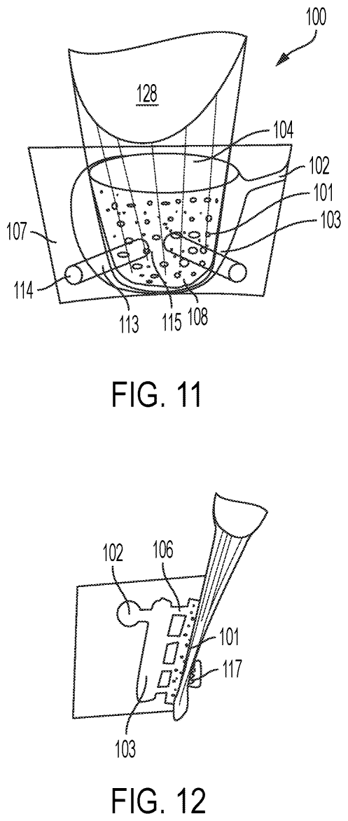

With reference to FIG. 11, the implant 109 may have a smooth surface 107 adjacent to the attachment surface 101 to reduce or eliminate unwanted attachment (i.e., grafting down) of tissue adjacent to the attachment surface 101. The attachment surface 101 may have a cross-section anatomically conforming to the connective tissue 104. Specifically, in the embodiment shown in FIG. 11, the attachment surface 101 may have a generally crescent moon shape, including a curved distal edge 108. This shaping of the attachment surface 101 allows specific attachment of the connective tissue 104 according to the shape of the distal end thereof, without necessarily exposing edges of the attachment surface 101 which may attach to surrounding tissue. The attachment surface 101 may further have anatomical curvature. For example, in the knee implant embodiment shown in FIG. 2, the attachment surface 101 may be curved around the main body 132 of the implant 109.

FIG. 11 shows an embodiment wherein the fastener comprises a pair of lateral and medial jaws 113, each having a fixed end 114 and a free end 115. The free end 115 may have teeth 116, as shown in FIG. 12. The fixed end 114 may be pivotable so that the jaw 113 can be rotated over the attachment surface 101 to secure the connective tissue 104 underneath.

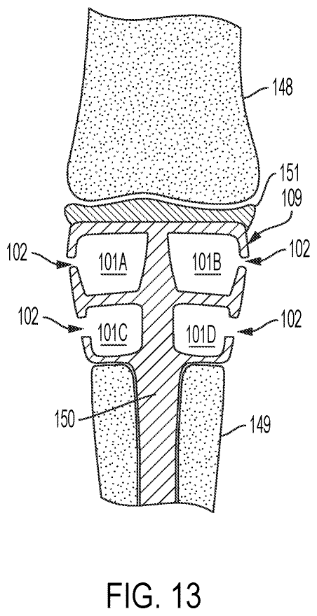

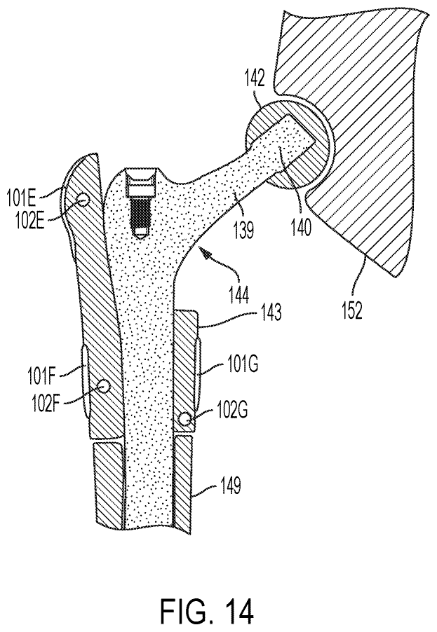

FIG. 13 shows an embodiment wherein the implant 109 is designed as a knee implant between the femur 148 and tibia 149 and which comprises an intramedullary stem which inserts into the tibia 149 and a condylar polymeric bearing block 151 interfacing the femur 148. In this embodiment, the implant 109 defines a plurality of attachment surfaces 101, each having an independent manifold 119 and suction port 102. As such, each attachment surface 101 may be operated independently (including being operated at different vacuum pressures) to induce suction at a position chosen for connective tissue 104 reattachment. In the embodiment shown in FIG. 13, the attachment surfaces 101 comprise a lateral attachment surface 101A and a medial attachment surface 101B. Furthermore, the attachment surfaces 101 may comprise a superior attachment surface 101A and an inferior attachment surface 101C. The specific embodiment shown in FIG. 14 comprises a quadrant of attachment surfaces 101 for the choice of connective tissue 104 reattachment both mediolaterally and superoinferiorly.

FIG. 13 shows an embodiment wherein the implant 109 comprises a quadrant of attachment surfaces 101 and respective ports 102 which may be individually utilised for the choice of connective tissue 104 reattachment position. In accordance with this embodiment, the at least one attachment surface 101 may be used for the reattachment of the extensor mechanism of the knee wherein the patellar tendon 141 is reattached to at least one of the attachment surfaces 101. The iliotibial tract may be attached to at least one of the lateral attachment surfaces 101A and 101C, and the sartorius, gracilis, and/or semitendinosus tendons may be attached to at least one of the medial attachment surfaces 101B and 101D.

FIG. 14 shows an embodiment wherein the implant 109 is a hip implant between the femur 149 and the 152. In accordance with this embodiment, the implant 109 may comprise a superior attachment surface 101E at the location of the greater trochanter and an associated suction port 102E. Approximately six different tendons of the hip may be attached to the superior attachment surface 101E. The attachment surface 101 may have anatomical curvature mimicking the anatomical profile of the greater trochanter. The implant 109 may further comprise a lateral attachment surface 101F and an associated suction port 102F. The lateral attachment surface 101F may be a continuation from the superior attachment surface 101E, and the implant 109 may comprise a matrix of channels 106 able to induce suction without substantial discontinuity between the superior attachment surface 101E and the lateral attachment surface 101F. The superior and lateral attachment surfaces 101E and 101F may be used for attachment of the illiofemoral ligament. The implant 109 may further comprise a medial attachment surface 101G and associated suction port 102G which may be used for attachment of the pubofemoral ligament.

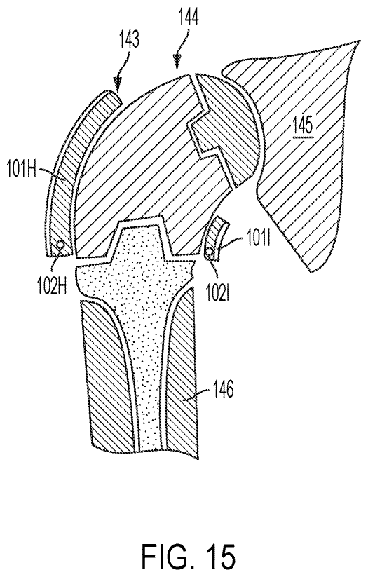

FIG. 15 shows the embodiment wherein the implant 109 is a shoulder implant 109 between the scapula 145 and the humorous 146 comprising a lateral attachment surface 101H and associated suction port 102H and a medial attachment surface 1011 and associated suction port 1021 which may be operated individually for attachment of the rotator cuff supraspinatus and infraspinatus muscle respectively.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A system for promoting biological attachment of fibrous connective tissue to a bone implant, the system comprising:
the bone implant comprising:
a scaffold defining a porous attachment surface;
a suction port; and
a manifold interfacing the scaffold and the suction port to distribute a negative pressure gradient across the attachment surface;
an extracorporeal vacuum pump;
a percutaneous suction hose interfacing the suction port of the implant and the vacuum pump, wherein, in use, the system is configured for:
the implant being implanted in a patient's body with the connective tissue applied over the attachment surface,
the suction hose being attached between the suction port and the vacuum pump outside the body;
the vacuum pump being operated outside the body for a period to induce the negative pressure gradient across the attachment surface to promote the biological integration of the connective tissue to the scaffold; and
the suction hose being detached from the suction port after the period.

2. The system as claimed in claim 1, further comprising a vascular tissue graft applied over the connective tissue, wherein the connective tissue is between the vascular tissue graft and the attachment surface.

3. The system as claimed in claim 2, wherein the is a vascular tissue graft is a pedicled muscle flap.

4. The system as claimed in claim 1, wherein the period is more than 1 week.

5. The system as claimed in claim 1, wherein the suction port is configured to disengage an inserted end of the suction hose when the suction hose is pulled.

6. The system as claimed in claim 5, wherein the suction port comprises a hose barb.

7. The system as claimed in claim 6, wherein the hose barb is entirely concealed within a recess.

8. The system as claimed in claim 1, wherein the manifold is defined by an internal chamber and a plurality of ducts interfacing the internal chamber and the scaffold.

9. The system as claimed in claim 1, wherein the attachment surface comprises a series of needles which engage the connective tissue.

10. The system as claimed in claim 9, wherein the needles are angled away with respect to a connective tissue pulling direction.

11. The system as claimed in claim 9, wherein the needles are permeable and define internal lumens forming part of the manifold.

12. The system as claimed in claim 11, wherein the needles comprise lumen interfacing side pores.

13. The system as claimed in claim 11, wherein the needles are cannulated.

14. The system as claimed in claim 1, wherein the implant comprises a main portion and a separable augment attachable to the main portion and wherein the augment comprises the manifold, the suction port and the scaffold.

15. The system as claimed in claim 14, wherein the augment defines an internal channel for engaging the main portion.

16. The system as claimed in claim 1, wherein the implant comprises a clamp configured to clamp across the connective tissue against the attachment surface.

17. The system as claimed in claim 16, wherein the clamp defines a manifold configured to distribute a negative pressure gradient across a permeable surface of the clamp.

18. The system as claimed in claim 16, wherein the clamp has an independent suction port.

19. The system as claimed in claim 1, wherein the attachment surface has a cross section anatomically conforming to the connective tissue.

20. The system as claimed in claim 19, wherein a distal edge of the attachment surface is curved.

21. The system as claimed in claim 19, wherein the implant has a smooth surface adjacent the attachment surface.

22. The system as claimed in claim 1, wherein the attachment surface has anatomical curvature.

23. The system as claimed in claim 1, wherein the implant comprises a main body defining a recess and wherein the scaffold is inserted and retained within the recess.

24. The system as claimed in claim 1, wherein the implant defines a plurality of attachment surfaces, each having respective suction ports.

25. The system as claimed in claim 1, wherein the implant is a knee implant.

26. The system as claimed in claim 25, wherein the attachment surface is configured to attach an extensor mechanism of the knee.

27. The system as claimed in claim 25, wherein the attachment surface is configured to attach a patellar connective tissue.

28. The system as claimed in claim 25, wherein the implant comprises a plurality of attachment surfaces and respective manifolds and wherein at least two of the attachment surfaces are configured to attach patellar, iliotibial tract sartorius, gracilis, and semitendinosus connective tissues respectively.

29. The system as claimed in claim 1, wherein the implant is a hip implant.

30. The system as claimed in claim 29, wherein the attachment surface is configured for attachment of the illiofemoral ligament.

31. The system as claimed in claim 30, wherein the attachment surface is laterally and superiorly located.

32. The system as claimed in claim 31, wherein the attachment surface has anatomical curvature mimicking an anatomical profile of a greater trochanter.

33. The system as claimed in claim 29, wherein the attachment surface is medially located and configured for attachment of the pubofemoral ligament.

34. The system as claimed in claim 29, wherein a gluteus maximus pedicled flap is applied over the connective tissue.

35. The system as claimed in claim 1, wherein the implant is a shoulder implant.

36. The system as claimed in claim 35, wherein the attachment surface is laterally located and configured for attachment of the supraspinatus muscle.

37. The system as claimed in claim 36, wherein the attachment surface is medially located and configured for attachment of the infraspinatus muscle.

38. A method involving the system as claimed in claim 1, the method comprising:
implanting the implant in a patient's body;
applying connective tissue over the attachment surface;
attaching the suction hose between suction port and the vacuum pump, the vacuum pump being outside the body;
operating the vacuum pump for a period to induce a negative pressure gradient across the attachment surface to promote the biological integration of the connective tissue to the scaffold; and
detaching the suction hose being detached from the suction port after the period.
